SEQUENCE LISTING (8 sequences)

SEQ ID NO: 1 - DNA, 21 bp, Artificial Sequence
  CD44 primer
atcctcacgtccaacaccctc

SEQ ID NO: 2 - DNA, 20 bp, Artificial Sequence
  CD44 primer
ctcgcctttcttggtgtagc

SEQ ID NO: 3 - DNA, 20 bp, Artificial Sequence
  CD29 primer
gatgccgggtttcactttgc

SEQ ID NO: 4 - DNA, 20 bp, Artificial Sequence
  CD29 primer
ttcccctgttccattcaccc

SEQ ID NO: 5 - DNA, 19 bp, Artificial Sequence
  OCT4 primer
gggacctcctagtgggtca

SEQ ID NO: 6 - DNA, 20 bp, Artificial Sequence
  OCT4 primer
tggcaaattgctcgaggtct

SEQ ID NO: 7 - DNA, 23 bp, Artificial Sequence
  Nanog primer
tcctcaatgacagatttcagaga

SEQ ID NO: 8 - DNA, 20 bp, Artificial Sequence
  Nanog primer
gagcaccaggtctgactgtt

(19) United States
(12) Patent Application Publication
(10) Pub. No.: US 2021/0284966 A1
(43) Pub. Date: Sep. 16, 2021

(54) METHOD OF DIFFERENTIATION INTO MESENCHYMAL STEM CELLS THROUGH CONTINUOUS SUBCULTURE OF DEDIFFERENTIATED STEM CELLS

(71) Applicant: KYUNGPOOK NATIONAL UNIVERSITY INDUSTRY-ACADEMIC COOPERATION FOUNDATION, Daegu (KR)

(72) Inventors: Kyu Shik JEONG, Daegu (KR); Hyun Sook PARK, Seoul (KR); Eun Joo LEE, Ulsan (KR); Sun Ray LEE, Seoul (KR); Yong Deuk KIM, Daegu (KR); Myung Jin CHUNG, Gyeongsan-si, Gyeongsangbuk-do (KR); Eun Mi LEE, Mungyeong-si, Gyeongsangbuk-do (KR); Sul Gi JEON, Daegu (KR)

(21) Appl. No.: 16/500,761

(22) PCT Filed: Apr. 3, 2018
(86) PCT No.: PCT/KR2018/003902

§ 371 Date: Oct. 3, 2019

(30) Foreign Application Priority Data
Apr. 4, 2017 (KR) 10-2017-0043781

(51) Int. Cl. C12N 5/0775

(52) U.S. Cl. CPC C12N 5/0667; C12N 2500/34; C12N 2500/38; C12N 2501/998; C12N 2501/165; C12N 2501/33

(57) ABSTRACT

The present invention relates to a medium for inducing differentiation of dedifferentiated stem cells into mesenchymal stem cells, a method for preparing mesenchymal stem cells from dedifferentiated stem cells by using the same, and mesenchymal stem cells prepared by using the same. The mesenchymal stem cells prepared using the above medium and method can be differentiated into various target cells, and thus can be useful as a cell therapeutic agent for congenital and acquired musculoskeletal diseases and injuries.

### TECHNICAL FIELD

The present disclosure is based on, and claims priority from, Korean Patent Application No. 10-2017-0043781, filed on Apr. 4, 2017, the disclosure of which is hereby incorporated by reference herein in its entirety.

The present disclosure relates to a medium for inducing differentiation of dedifferentiated stem cells into mesenchymal stem cells, a method of preparing mesenchymal stem cells from dedifferentiated stem cells using the medium, and mesenchymal stem cells prepared by using the same.

### BACKGROUND ART

Dedifferentiated stem cells are cells having pluripotency, and their differentiation into three germ layers of ectoderm, mesoderm, and endoderm is possible. Such pluripotency is the greatest advantage of dedifferentiated stem cells. However, to practically use dedifferentiated stem cells for clinical and drug screening, it is required to differentiate them into target cells. To reduce the carcinogenic risk which is pointed out as the biggest problem of dedifferentiated stem cells, it is also required to develop a differentiation medium or a differentiation method capable of stably differentiating dedifferentiated stem cells.

Therefore, differentiation methods of differentiating dedifferentiated stem cells into various target cells have been suggested. However, when dedifferentiated stem cells are differentiated, there is a difference in the differentiation probabilities between the three germ layers. When dedifferentiated stem cells are differentiated into each of three germ layers, the probability of differentiation into mesoderm is lower than the probability of differentiation into endoderm and ectoderm, and it is the most difficult to differentiate dedifferentiated stem cells into mesoderm. Therefore, use of various small molecule compounds has been introduced to promote differentiation into mesoderm.

Dedifferentiated stem cells derived from various animals, including humans, have been established. Among them, equine dedifferentiated stem cells have similar characteristics to human dedifferentiated stem cells. Maintenance and differentiation of human and equine dedifferentiated stem cells are more difficult than those of mouse dedifferentiated stem cells, and research is needed to overcome this difficulty. In addition, since dedifferentiated stem cells have pluripotency, differentiation of dedifferentiated stem cells may lead to differentiation into unwanted cells. This is a problem that is required to be overcome in the study and practical application of dedifferentiated stem cells. In the same context, purity or homogeneity of cells obtained after differentiating dedifferentiated stem cells is important.

In view of the above, the present inventors have made intensive efforts to differentiate dedifferentiated stem cells into mesenchymal stem cells, and as a result, they found that dedifferentiated stem cells may be differentiated into mesenchymal stem cells having excellent proliferative capacity, thereby completing the invention

### DESCRIPTION OF EMBODIMENTS

### Technical Problem

An aspect provides a medium for inducing differentiation of dedifferentiated stem cells into mesenchymal stem cells, the medium including glucose, insulin, selenium, transferrin, and vascular endothelial growth factor (VEGF).

Another aspect provides a method of preparing mesenchymal stem cells from dedifferentiated stem cells, the method including introducing a dedifferentiation inducer protein or a polynucleotide encoding the same into isolated somatic cells or isolated adult stem cells to induce dedifferentiation of dedifferentiated stem cells from the isolated somatic cells or the isolated adult stem cells; and culturing the induced dedifferentiated stem cells in the medium for inducing differentiation to induce differentiation of mesenchymal stem cells from dedifferentiated stem cells.

Still another aspect provides mesenchymal stem cells prepared by the above method.

### Solution to Problem

An aspect provides a medium for inducing differentiation of dedifferentiated stem cells into mesenchymal stem cells.

The “dedifferentiation” refers to a process by which differentiated cells may be restored in a state having a new type of differentiation potential. Further, the dedifferentiation may be used in the same sense as cell reprogramming. Such a cell dedifferentiation or reprogramming mechanism establishes a different set of epigenetic marks after the deletion of epigenetic marks in the nucleus (DNA state associated with causing a genetic change in a function without a change in a nucleotide sequence). The ‘dedifferentiation’ may include any process of returning the differentiated cells having differentiation capacity of 0% to less than 100% into an undifferentiated state, for example, a process of restoring or converting differentiated cells having differentiation capacity of 0%, or partially differentiated cells having differentiation capacity of more than 0% to less than 100% into cells having differentiation capacity of 100%.

The “dedifferentiated stem cells” have the same meaning as “induced pluripotent stem cells (iPSCs)”, and may refer to induced pluripotent stem cells produced by reprogramming somatic cells or adult stem cells by expression of reprogramming factors or by inducing the expression thereof.

The “mesenchymal stem cells (MSCs)” may refer to stem cells having multipotency and self-renewal capacity, which may be differentiated into various cells such as adipocytes, chondrocytes, and osteoblasts.

The medium for inducing differentiation may be to induce differentiation of dedifferentiated stem cells into mesenchymal stem cells. The mesenchymal stem cells may have surface antigenic characteristics of CD29⁺ and/or CD44⁺. That is, when dedifferentiated stem cells are differentiated/proliferated, at least about 20%, about 25%, about 30%, about 35%, about 40%, about 45%, about 50%, about 55%, about 60%, about 65%, about 70%, about 75%, about 80%, about 85%, about 90%, about 95%, about 98%, or about 99% of the mesenchymal stem cells may express CD29 and/or CD44 on the cell surface thereof. The mesenchymal stem cells may be positive for CD29 and/or CD44 on the cell surface thereof. The term “positive”, in reference to a stem cell marker, may mean that the marker is present in a higher amount or in a higher level than that of the marker in a reference other cell. In other words, a cell is positive for a marker when the cell may be distinguished from one or more other cell types on the basis of the presence of the marker inside the cell or on the surface of the cell. The term “negative” may mean that even when an antibody specific to a specific cell surface marker is used, the marker is not detected, as compared with a background value. The above characteristics may be determined by methods commonly used in the art. For example, various methods such as flow cytometry, immunohistochemical staining, RT-PCR, etc. may be used.

The medium may include glucose, insulin, selenium, transferrin, and vascular endothelial growth factor (VEGF).

The glucose is a kind of sugar, has an effect of providing an energy source necessary for cell division or differentiation, and may have a molecular formula of C₆H₁₂O₆. The glucose may be included in an amount of 100 mg/L to 10000 mg/L, 200 mg/L to 5000 mg/L, 500 mg/L to 2000 mg/L, 750 mg/L to 1500 mg/L, 900 mg/L or 1100 mg/L, or 1000 mg/L (μg/ml) in the medium.

The insulin is a hormone that maintains a constant amount of glucose in the blood and has an effect of promoting cell division or differentiation. The insulin may be included in an amount of 0.3 mg/L to 30 mg/L, 0.6 mg/L to 15 mg/L, 1.5 mg/L to 6 mg/L, 3 mg/L to 5 mg/L, 2 mg/L to 5 mg/L, or 3 mg/L (μg/ml) in the medium.

The selenium is a substance that allows selenium-dependent enzymes to reduce the peroxidation of lipids, and has an antioxidant power. The selenium may be included in an amount of 0.0000003 mg/L to 0.00003 mg/L, 0.0000006 mg/L to 0.000015 mg/L, 0.0000015 mg/L to 0.000006 mg/L, 0.000002 mg/L to 0.000004 mg/L, or 0.000003 mg/L (μg/ml) in the medium.

The selenium may be selenium itself, and a salt of selenium, for example, an organic or inorganic form. The organic form of the selenium salt may be amino acid L(+)-selenomethionine, L(+)-methylselenocysteine, or L(+)-selenocysteine. The inorganic form of the selenium salt may be sodium selenite, calcium selenite, or potassium selenite. The selenium, for example, sodium selenite may be included in an amount of 0.0000003 mg/L to 0.00003 mg/L, 0.0000006 mg/L to 0.000015 mg/L, 0.0000015 mg/L to 0.000006 mg/L, 0.000002 mg/L to 0.000004 mg/L, or 0.000003 mg/L (μg/ml) in the medium.

The transferrin is a glycoprotein in which trivalent iron in the blood binds to globulin in plasma proteins, and has an effect of transferring irons to cells. The transferrin may be included in an amount of 0.27 mg/L to 27 mg/L, 0.54 mg/L to 13.5 mg/L, 1.35 mg/L to 5.4 mg/L, 2.2 mg/L to 3.2 mg/L, or 2.7 mg/L (μg/ml) in the medium.

The VEGF has an effect of promoting cell division or differentiation, and the VEGF may be included in an amount of 0.001 mg/L to 0.1 mg/L, 0.002 mg/L to 0.05 mg/L, 0.005 mg/L to 0.02 mg/L, 0.0075 mg/L to 0.015 mg/L, or 0.01 mg/L (μg/ml) in the medium.

The medium for inducing differentiation may include vitamin B.

The medium for inducing differentiation may include biotin (coenzyme R) and niacin (nicotinic acid, nicotinamide, niacinamide). The biotin and niacin are a kind of vitamin B, have an effect of maturing dedifferentiated stem cells in an undifferentiated state into mesenchymal stem cells, and may have a molecular formula of C₁₀H₁₆N₂O₃s and C₆H₅NO₂, respectively. The biotin may be included in an amount of 0.01 mg/L to 1 mg/L, 0.02 mg/L to 0.5 mg/L, 0.05 mg/L to 0.2 mg/L, 0.075 mg/L to 0.15 mg/L, or 0.1 mg/L (μg/ml) in the medium, and the niacin may be included in an amount of 0.1 mg/L to 10 mg/L, 0.2 mg/L to 5 mg/L, 0.5 mg/L to 2 mg/L, 0.75 mg/L to 1.5 mg/L, or 1 mg/L (μg/ml) in the medium. The biotin and the niacin may be included at a weight ratio of 1:20 to 1:5, 1:15 to 1:7, or 1:10.

The medium for inducing differentiation may include one or more selected from the group consisting of thiamine (vitamin B1), riboflavin (vitamin B2), pantothenic acid (vitamin B5), pyridoxal (vitamin B6), folic acid (vitamin B9), and cobalamin (vitamin B12). The thiamine may be, for example, thiamine HCl. The pantothenic acid may be, for example, a D-Ca pantothenate. The pyridoxal may be, for example, pyridoxal HCl. The medium for inducing differentiation may further include one or more selected from the group consisting of ascorbic acid, choline, and inositol. The ascorbic acid may be, for example, L-ascorbic acid. The choline may be, for example, choline chloride. The inositol may be i-inositol.

The thiamine, riboflavin, pantothenic acid, pyridoxal, folic acid, and cobalamin, and ascorbic acid, choline and inositol may be included in an amount of 0.1 mg/L to 80 mg/L, respectively. The pantothenic acid (e.g., D-Ca pantothenate), choline (e.g., choline chloride), folic acid, and pyridoxal (e.g., pyridoxal HCl) may be included in an amount of 0.1 mg/L to 80 mg/L, 0.1 mg/L to 10 mg/L, 0.2 mg/L to 5 mg/L, 0.5 mg/L to 2 mg/L, 0.75 mg/L to 1.5 mg/L, or 1 mg/L (μg/ml) in the medium, respectively. The ascorbic acid (e.g., L-ascorbic acid) may be included in an amount of 6.5 mg/L to 650 mg/L, 13 mg/L to 320 mg/L, 33 mg/L to 130 mg/L, 50 mg/L to 90 mg/L, 60 mg/L to 80 mg/L, 65 mg/L to 70 mg/L, or 67 mg/L (μg/ml) in the medium. The inositol (e.g., i-inositol) may be included in an amount of 0.2 mg/L to 20 mg/L, 0.4 mg/L to 10 mg/L, 1 mg/L to 4 mg/L, 1.5 mg/L to 3 mg/L, or 2 mg/L (μg/ml) in the medium. The riboflavin may be included in an amount of 0.01 mg/L to 1 mg/L, 0.02 mg/L to 0.5 mg/L, 0.05 mg/L to 0.2 mg/L, 0.075 mg/L to 0.15 mg/L, or 0.1 mg/L (μg/ml) in the medium. The thiamine (e.g., thiamine HCl) may be included in an amount of 0.4 mg/L to 40 mg/L, 0.8 mg/L to 20 mg/L, 2 mg/L to 8 mg/L, 3 mg/L to 5 mg/L, or 4 mg/L (μg/ml) in the medium. The vitamin B12 may be included in an amount of 0.14 mg/L to 14 mg/L, 0.28 mg/L to 7 mg/L, 0.7 mg/L to 2.8 mg/L, 1.05 mg/L to 2.1 mg/L, or 1.4 mg/L (μg/ml) in the medium.

The medium for inducing differentiation may include ribonucleoside and/or deoxyribonucleoside. The ribonucleoside may include one or more selected from the group consisting of adenosine, cytidine, guanosine, and uridine. The deoxyribonucleoside may include one or more selected from the group consisting of deoxyadenosine (e.g., 2′deoxyadenosine), deoxycytidine (e.g., 2′deoxycytidine.HCl), deoxyguanosine (e.g., 2′deoxyguanosine), and thymidine. The adenosine, cytidine, guanosine, uridine, deoxyadenosine (e.g., 2′deoxyadenosine), deoxycytidine (e.g., 2′deoxycytidine.HCl), deoxyguanosine (e.g., 2′deoxyguanosine), and thymidine may be included in an amount of 1 mg/L to 100 mg/L, 2 mg/L to 50 mg/L, 5 mg/L to 20 mg/L, 7.5 mg/L to 15 mg/L, or 10 mg/L (μg/ml) in the medium.

Within the above ranges, the above substances may further promote differentiation of dedifferentiated stem cells into mesenchymal stem cells.

The glucose, insulin, selenium, transferrin, VEGF, biotin, niacin, etc. may be isolated from a natural source or may be prepared by a chemical synthetic method.

A method of transferring the medium for inducing differentiation to dedifferentiated stem cells may be to contact the dedifferentiated stem cells with the composition. The contact may be, for example, to culture the dedifferentiated stem cells in the medium for inducing differentiation.

The medium for inducing differentiation may include amino acids. The amino acids may be provided as oxidation nutrients or metabolites. The medium for inducing differentiation may include, for example, one or more selected from the group consisting of glycine, L-alanine, L-asparagine, L-aspartate, L-cysteine, L-glutamate, L-glutamine, L-histidine, L-hydroxyproline, L-isoleucine, L-leucine, L-lysine, L-methionine, L-phenylalanine, L-proline, L-serine, L-threonine, L-tryptophan, L-tyrosine, L-arginine, L-valine, and L-taurine. The amino acids may be included in an amount of 5 mg/L to 300 mg/L, respectively.

The medium for inducing differentiation may include a medium commonly used in cell culture or a prepared medium suitable for differentiation into mesenchymal stem cells. The medium used in cell culture may generally include a carbon source, a nitrogen source, and trace elements. The medium used in cell culture may include, for example, one or more selected from the group consisting of Dulbecco's Modified Eagle's Medium (DMEM), Minimal Essential Medium (MEM), Basal Medium Eagle (BME), RPMI 1640, F-10, F-12, DMEM/F12, α-Minimal Essential Medium (α-MEM), Glasgow's Minimal Essential Medium (G-MEM), Iscove's Modified Dulbecco's Medium (IMDM), MacCoy's 5A medium, AmnioMax complete medium, AminoMaxil complete medium, Chang's Medium, and MesenCult-XF.

The medium for inducing differentiation may include an animal-derived serum. The serum may be one or more selected from the group consisting of fetal bovine serum (FBS) and bovine calf serum (BCS). The serum may be used in a volume of 0.5% to 50%, 1% to 25%, 2.5% to 12.5%, 3.5% to 6.5%, or 5% with respect to the total volume of the medium for inducing differentiation.

In addition, the medium for inducing differentiation may include an antibiotic agent, an antifungal agent, and a reagent to prevent mycoplasma growth. The antibiotic agent may be, for example, penicillin, streptomycin, or fungizone. The antifungal agent may be, for example, amphotericin B. The mycoplasma inhibitor may be, for example, tylosin. To prevent mycoplasma contamination, for example, gentamicin, ciprofloxacin, azithromycin, etc. may be used.

The dedifferentiated stem cells may be those derived from a mammal, for example, a horse, a dog, a cat, a fetus, a calf, a human, or a mouse. The dedifferentiated stem cells may be those derived from an adipose tissue, a bone marrow, umbilical cord blood, or a placenta of a mammal, for example, a horse, a dog, a cat, a fetus, a calf, a human, or a mouse.

Another aspect provides a method of preparing mesenchymal stem cells from dedifferentiated stem cells, the method including introducing a dedifferentiation inducer protein or a polynucleotide encoding the same into isolated somatic cells or isolated adult stem cells to induce dedifferentiation of dedifferentiated stem cells from the isolated somatic cells or the isolated adult stem cells; and culturing the induced dedifferentiated stem cells in the medium for inducing differentiation to induce differentiation of mesenchymal stem cells from dedifferentiated stem cells.

The “isolated” may mean cells existing in an environment different from a naturally occurring intracellular environment. For example, when cells naturally occur in a multicellular organ and the cells are removed from the multicellular organ, the cells are those “isolated”.

The “somatic cell” may mean a cell constituting an adult, of which differentiation capacity and self-renewal capacity are limited. The somatic cell may be an adipocyte, a bone marrow, umbilical cord blood, a placenta, a nerve, a muscle, a skin, a hair, etc. of a mammal, for example, a horse, a dog, a cat, a fetus, a calf, a human, or a mouse. For example, the somatic cell may be an adipocyte of a horse or a human.

The “adult stem cells” refer to stem cells that appear in the stage of formation of each organ of an embryo during development or in the adult stage, and its differentiation capacity is generally limited to only cells constituting specific tissues. The adult stem cells may be nerve stem cells that are able to differentiate into neurons, hematopoietic stem cells that are able to differentiate into blood cells, mesenchymal stem cells that are able to differentiate into bone, cartilage, fat, and muscle, and hepatic stems that are able to differentiate into hepatocytes. The adult stem cells may maintain proliferative capacity, may be advantageous in securing an effective number of cells that may be induced into dedifferentiated stem cells, and may have high induction efficiency into dedifferentiated stem cells, as compared with somatic cells. The adult stem cells may be, for example, mesenchymal stem cells, and may be derived from an adipose tissue, a bone marrow, umbilical cord blood, or a placenta of a mammal, for example, a horse, a dog, a cat, a fetus, a calf, a human, or a mouse. When adult stem cells are mesenchymal stem cells derived from an adipose tissue of a human or a horse, a large amount thereof may be relatively easily provided, unlike stem cells of a bone marrow, umbilical cord blood, or a placenta. Since about 1% of adipocytes is estimated to be stem cells, it is advantageous in that the yield is high. With regard to mesenchymal stem cells derived from an adipose tissue of a human or a horse, autologous stem cells may be used, which is less likely to cause immune rejection.

The isolated somatic cells or the isolated adult stem cells may be obtained by methods commonly used in the art. The isolated somatic cells or the isolated adult stem cells may be obtained by, for example, cutting an adipose tissue, a bone marrow, umbilical cord blood, or a placenta into many pieces using sterile scissors. With regard to a placenta, for example, placenta somatic cells or placenta adult stem cells may be obtained by attaching the placenta to a culture plate and culturing the placenta, confirming that the cells extend from the separated placenta, reacting the cells with a separating enzyme, filtering the cells through a cell strainer, and centrifuging the cells. With regard to an adipose tissue, for example, the cells may be obtained by reacting the adipose tissue with a separating enzyme, filtering the cells through a cell strainer, and centrifuging the cells. The separating enzyme may include collagenase. The collagenase may refer to an enzyme that breaks peptide bonds of collagen, and may include collagenase type I, type II, type III, type IV, or a combination thereof.

The “dedifferentiation inducer” is a factor used to reprogram somatic cells or adult stem cells into dedifferentiated stem cells, and may be derived from a mammal such as a horse, a dog, a cat, a fetus, a calf, a human, or a mouse. For example, the dedifferentiation inducer may be one or more selected from the group consisting of Oct4 (also referred to as Oct3/4), Sox2, KIF4, c-Myc, Nanog, and Lin-28 Each protein of Oct4, Sox2, KIF4, c-Myc, Nanog, and Lin-28 may be a protein having an amino acid sequence of a wild-type thereof, or may be altered by substitution, deletion, insertion of one or more amino acids, or a combination thereof. Each polynucleotide encoding each protein of Oct4, Sox2, KIF4, c-Myc, Nanog, and Lin-28 may be a nucleotide sequence encoding the wild-type protein thereof, or may be altered by substitution, deletion, insertion of one or more bases, or a combination thereof. Each amino acid sequence or nucleotide sequence of Oct4, Sox2, KIF4, c-Myc, Nanog, and Lin-28 may be identified with reference to NCBI (http://www.ncbi.nlm.nih.gov). Further, the polynucleotide may be isolated from a natural source or may be prepared by a chemical synthetic method.

The method may include introducing the dedifferentiation inducer protein or the polynucleotide encoding the same into isolated somatic cells or isolated adult stem cells to induce dedifferentiation of dedifferentiated stem cells from the isolated somatic cells or the isolated adult stem cells.

The introducing of the dedifferentiation inducer protein or the polynucleotide encoding the same into isolated somatic cells or isolated adult stem cells may be to express one or more reprogramming factors in the somatic cells or the adult stem cells. The somatic cells or the adult stem cells may be reprogrammed by expressing one reprogramming factor, at least two reprogramming factors, at least three reprogramming factors, at least four reprogramming factors, or five reprogramming factors. The reprogramming factor may be selected from the group consisting of Oct4, Sox2, KIF4, c-Myc, Nanog, and Lin-28. The somatic cells or the adult stem cells may be reprogrammed by expressing at least one, two, three, four or five reprogramming factors. The reprogramming factor may be an exogenous nucleic acid encoding the same. The exogenous nucleic acid encoding the reprogramming factor may be increased in its expression, as compared with cells that are not genetically modified. The increased expression may be caused by introducing the exogenous nucleic acid encoding the reprogramming factor into cells.

The expression of the reprogramming factor may be induced by contacting somatic cells or adult stem cells with at least one substance such as a small organic molecule inducing expression of the reprogramming factor. The somatic cells or the adult stem cells may also be reprogrammed by a combination of expressing reprogramming factors (e.g., using viral vectors, plasmids, etc.) and inducing expression of the reprogramming factors (e.g., using small organic molecules). The reprogramming factors may be expressed in somatic cells or adult stem cells by infection with a viral vector, for example, a retroviral vector, a lentiviral vector, or a sendai viral vector. Further, the reprogramming factors may be expressed in somatic cells or adult stem cells using a non-integrating vector, for example, an episomal vector (see Yu et al., Science. 2009 May 8; 324(5928):797-801). When the reprogramming factors are expressed using the non-integrating vector, the factors may be expressed by electroporation, transfection, lipofection, or transformation.

Once the reprogramming factors are expressed in cells, the cells may be cultured. After introducing the dedifferentiation inducer protein or the polynucleotide encoding the same into isolated somatic cells or isolated adult stem cells, the cells may be cultured for 15 days or more, 16 days or more, 18 days or more, 20 days or more, 25 days or more, 30 days or more, 35 days or more, 40 days or more, or 15 days to 40 days, 16 days to 35 days, 18 days to 30 days. At this time, the medium may include, for example, DMEM, FBS, GlutaMAX, MEM-NEAA, penicillin/streptomycin, LIF, mercaptoethanol, doxycycline, or a combination thereof. The doxycycline may be included in an amount of 0.5 mg/L to 5 mg/L, 1 mg/L to 3 mg/L, or about 2 mg/L (μg/ml) in the medium. Cells with characteristics of embryonic stem cells may appear on a culture dish over time.

Particular dedifferentiated stem cells may vary in their expression profiles, but may be commonly identified by expression of the same markers as in embryonic stem cells. Cells that appear on a culture dish may be selected and subcultured, for example, based on morphology of embryonic stem cells or based on expression of selectable and detectable markers.

To confirm the pluripotency of dedifferentiated stem cells, the cells may be tested by one or more pluripotency assays. For example, cells may be examined for expression of embryonic stem cell markers; cells may be assessed for their ability to produce teratomas upon transplantation into a severe combined immunodeficient (SCID) mouse; and assessed for the differentiation ability to produce cell types of all three germ layers. In addition, cells may be assessed for expression levels of, for example, Oct4, alkaline phosphatase (AP), SSEA 3 surface antigen, SSEA 4 surface antigen, TRA 1 60, and/or TRA 1 81.

After culturing the somatic cells or the adult stem cells into which reprogramming factors have been introduced, the cells may be cultured using feeder cells to grow dedifferentiated stem cells. The term “feeder cells”, also referred to as support cells, mean cells which are pre-cultured to play a role in supplying nutrients or proliferation factors insufficient in the medium, when cells that are not able to survive alone are cultured. The cells may also be proliferated by a known method without using feeder cells, thereby preventing contamination of the feeder cells during clinical application of dedifferentiated stem cells.

A method of recovering the dedifferentiated stem cells may be performed by using a separating enzyme which is applicable in a general method of culturing dedifferentiated stem cells. For example, the medium may be removed from the culture dish on which the dedifferentiated stem cells are cultured, the dish may be washed with PBS once, a solution containing an appropriate separating enzyme (collagenase, trypsin, dispase, or a combination thereof) may be added thereto, the cells may be allowed to react with the separating enzyme, and then suspended to recover single cells.

The method may include culturing the induced dedifferentiated stem cells in the medium for inducing differentiation to induce differentiation of dedifferentiated stem cells into mesenchymal stem cells.

The medium for inducing differentiation may include glucose, insulin, selenium, transferrin, and VEGF. The medium for inducing differentiation may include biotin and niacin. The medium for inducing differentiation is the same as described above.

A method of transferring the medium for inducing differentiation to dedifferentiated stem cells may be to contact the dedifferentiated stem cells with the medium. The contact may be, for example, to culture the dedifferentiated stem cells in the medium for inducing differentiation. The method may be to continue subculture by proliferating the cells in the medium for inducing differentiation of dedifferentiated stem cells into mesenchymal stem cells. Through the continuous subculture, it is possible to remove undifferentiated cells and aged cells. Through the continuous subculture, it is possible to obtain mesenchymal stem cells having excellent morphological and/or surface antigenic characteristics.

In the subculture, a separating enzyme may be used. The separating enzyme may be an enzyme that degrades intercellular binding proteins. The subculture may be performed by, for example, washing the culture plate with PBS once when the cultured cells occupy about 60% to about 100%, about 70% to about 100%, or about 80% to about 90% of the area of the culture plate, adding an appropriate separating enzyme thereto, reacting the cells with the separating enzyme, suspending the cells, and seeding the cells in another culture plate. The separating enzyme may include an enzyme that degrades intercellular binding proteins, which is commonly used for subculture in the art, and an enzyme that degrades binding proteins, which is known to those skilled in the art, may be used after appropriate modification. The enzyme that degrades intercellular binding proteins may be, for example, TrypLE™ Select (GIBCO Invitrogen), TrypLE™ Express (GIBCO Invitrogen), TrypZean™ (Sigma Aldrich), or Recombinant Trypsin Solution™ (Biological Industries).

The method may be to subculture the induced dedifferentiated stem cells in the medium for inducing differentiation for 1 passage to 25 passages, 1 passage to 18 passages, 2 passages to 18 passages, 2 passages to 14 passages, 3 passages to 14 passages, 4 passages to 14 passages, 3 passages to 10 passages, 4 passages to 9 passages, 5 passages to 9 passages, 6 passages to 8 passages, or 7 passages.

The method may be to culture the induced dedifferentiated stem cells in the medium for inducing differentiation for 2 days to 80 days, 5 days to 75 days, 10 days to 70 days, 15 days to 70 days, 20 days to 70 days, 22 days to 70 days, 25 days to 60 days, 27 days to 50 days, 30 days to 40 days, 33 days to 37 days, or 35 days.

Still another aspect provides mesenchymal stem cells prepared by the above method.

The mesenchymal stem cells may have surface antigenic characteristics of CD29⁺ and CD44⁺. In other words, the mesenchymal stem cells may have surface antigenic characteristics of CD29⁺ and/or CD44⁺. The mesenchymal stem cells are the same as described above.

The mesenchymal stem cells prepared by the above method may have continuously high proliferative capacity and differentiation capacity. Therefore, it is possible to subculture the mesenchymal stem cells prepared by the above method up to 25 passages while maintaining characteristics of mesenchymal stem cells.

Even though the mesenchymal stem cells induced from the dedifferentiated stem cells are repeatedly subcultured, they exhibit excellent proliferative capacity, as compared with mesenchymal stem cells of adult stem cells, and thus there is a remarkable difference in terms of the quantity of mesenchymal cells. Even though repeatedly subcultured, the mesenchymal stem cells may maintain their morphological characteristics, and may express surface markers of mesenchymal stem cells, and therefore, in terms of quality, the mesenchymal stem cells may continuously maintain their characteristics.

### Advantageous Effects of Disclosure

According to a medium for inducing differentiation of dedifferentiated stem cells into mesenchymal stem cells and a method of preparing mesenchymal stem cells from dedifferentiated stem cells using the same, mesenchymal stem cells having excellent proliferative capacity may be obtained, and therefore, it is easy to obtain a sufficient number of cells needed for cell therapy. Further, through continuous subculture, mesenchymal stem cells having high purity may be obtained, and thus the mesenchymal stem cells are safe enough to be used as a cell therapeutic agent. The mesenchymal stem cells prepared using the medium and the method may differentiate into various target cells such as muscles, tendons, ligaments, bones, etc., thereby being usefully applied to cell therapeutic agents for congenital and acquired musculoskeletal diseases and injuries.

### BRIEF DESCRIPTION OF DRAWINGS

FIG. 1A shows optical microscopy images of a differentiation process of stem cells dedifferentiated into mesenchymal stem cells, wherein DT represents a period of differentiation into mesenchymal stem cells, and P represents a passage number; FIG. 1B shows optical microscopy images of differentiated stem cells at 7 passages and 35 days post-differentiation of stem cells dedifferentiated into mesenchymal stem cells; FIG. 1C shows optical microscopy images of differentiated stem cells at 14 passages and 70 days post-differentiation of stem cells dedifferentiated into mesenchymal stem cells;

FIG. 2 shows real-time PCR (RT-PCR) results of examining mRNA levels of CD44 and CD29 in equine dedifferentiated stem cells (equine induced pluripotent stem cell: E-iPS), equine adipose-derived mesenchymal stem cells (E-ASC), and mesenchymal stem cells differentiated from equine dedifferentiated stem cells (differentiated mesenchymal stem cells derived from equine induced pluripotent stem cell: Df-E-iPS);

FIG. 3 shows RT-PCR results of examining mRNA levels of OCT4 and Nanog in equine dedifferentiated stem cells (E-iPS), equine adipose-derived mesenchymal stem cells (E-ASC), and mesenchymal stem cells differentiated from equine dedifferentiated stem cells (Df-E-iPS); and

FIG. 4 shows fluorescence activated cell sorting (FACS) results of examining expression of cell surface markers CD44 and CD29 in equine dedifferentiated stem cells (E-iPS), equine adipose-derived mesenchymal stem cells (E-ASC), and mesenchymal stem cells differentiated from equine dedifferentiated stem cells (Df-E-iPS).

### BEST MODE

Hereinafter, preferred examples will be provided for better understanding of the present disclosure. However, the following examples are provided only for understanding the present disclosure more easily, but the content of the present disclosure is not limited thereby.

### Example 1. Preparation of Dedifferentiated Stem Cells and Induction of Differentiation of Dedifferentiated Stem Cells into Mesenchymal Stem Cells Using Medium for Inducing Differentiation

1. Preparation of Dedifferentiated Stem Cells from Equine Adipose Tissue

An adipose tissue collected from an 8-month-old horse was washed with Dulbecco's Phosphate-buffered saline (DPBS) (GeneDEPOT) and 70% ethanol (Duksan Pure Chemicals). The adipose tissue was minced using a razor blade, and put in PBS containing 0.2% type I collagenase (Worthington Biochemical), and allowed to digest in an incubator at 37° C. for 10 minutes. The digested tissue was passed through a 70-μm nylon cell strainer (SPL Life Sciences), and a cell pellet was resuspended and washed with PBS to isolate equine adipose tissue-derived adult stem cells. The isolated equine adipose tissue-derived stem cells were incubated in a medium containing low-glucose Dulbecco's modified Eagle's medium (DMEM), 10% fetal bovine serum (FBS), and 1% penicillin/streptomycin under conditions of 37° C. and 5% CO₂. The equine adipose tissue-derived adult stem cells were subcultured once. At 1^{st} passage (1 day prior to transduction), 1×10⁵ cells were seeded on a 100 mm dish coated with 0.1% gelatin.

For transduction of Yamanaka factors Oct4, Sox2, KIF4, and c-Myc using lentivirus, 293FT cell line was transduced with TetO-FUW-OSKM plasmid (ADDGENE #20321) or FUW-M2rtTA plasmid (ADDGENE #20342) using a Virapower packaging mix (Invitrogen) according to the manufacturer's instructions. Thereafter, the supernatant was collected and filtered with a 0.45 μm filter (Millipore) to remove cell debris. 10 μg/ml polybrene (Sigma) was added thereto, followed by transduction for 24 hours. After completing transduction, the medium was replaced by a medium containing high glucose DMEM, 10% FBS, and penicillin/streptomycin, followed by incubation for 24 hours. Thereafter, the transduced adipose tissue-derived adult stem cells were transferred onto a feeder, of which growth was inhibited with mitomycin C, and cultured for 30 days in a medium (hereinafter, referred to as ESC medium) containing high glucose DMEM, 20% FBS, 1% GlutaMAX, 1% MEM-non-essential amino (MEM-NEAA), 1% penicillin/streptomycin, leukemic inhibitory factor (LIF) (1000 units/ml), and 0.1% mercaptoethanol, the medium mixed with 2 μg/ml doxycycline, while replacing this medium every other day. At 18 days or 30 days post-transduction, colonies having morphology similar to human embryonic stem cells were collected from the ESC medium, and transferred onto a new feeder and subcultured in ESC medium containing 2 μg/ml doxycycline to prepare equine dedifferentiated stem cells.

2. Induction of Differentiation of Dedifferentiated Stem Cells into Mesenchymal Stem Cells Using Medium for Inducing Differentiation of Mesenchymal Stem Cells (MSCs)

The equine dedifferentiated stem cells established in 1 were detached from the culture plate by reacting with a 10 mg/mL type IV collagenase solution at 37° C. for 10 minutes, and seeded at a density of 1×10⁴ cells/cm² on a 100 mm dish coated with 0.1% gelatin. A basic medium was mixed with 3 mg/L insulin, 0.000003 mg/L sodium selenite, 2.7 mg/L transferrin, 0.01 mg/L VEGF, 0.1 mg/L biotin, 1 mg/L niacinamide, and 1000 mg/L D-glucose, and FBS was added at a volume of 5% with respect to the total volume to prepare a medium for inducing MSC differentiation (hereinafter, referred to as a medium for inducing MSC differentiation)

TABLE 1
Basic medium
Inorganic salts (mg/L)

| | |
|---|---|
| CaCl₂ (anhyd.) | 200 |
| KCl | 400 |
| MgSO₄ (anhyd.) | 98 |
| NaCl | 6,500 |
| NaH₂PO₄H₂O | 140 |

Vitamin (mg/L)

| | |
|---|---|
| L-ascorbic acid | 67 |
| D-Ca pantothenate | 1 |
| Choline chloride | 1 |
| Folic acid | 1 |
| i-inositol | 2 |
| Pyridoxal HCl | 1 |
| Riboflavin | 0.1 |
| Thiamine HCl | 4 |
| Vitamin B12 | 1.4 |

Ribonucleoside (mg/L)

| | |
|---|---|
| Adenosine | 10 |
| Cytidine | 10 |
| Guanosine | 10 |
| Uridine | 10 |

Deoxyribonucleoside (mg/L)

| | |
|---|---|
| 2′Deoxyadenosine | 10 |
| 2′Deoxycytidine••HCl | 10 |
| 2′Deoxyguanosine | 10 |
| Thymidine | 10 |

Other components (mg/L)

| | |
|---|---|
| AlbuMAX 1 | 4000 |
| Lipoic acid | 0.2 |
| Reduced glutathione | 0.5 |
| Sodium pyruvate | 110 |

The equine dedifferentiated stem cells were differentiated and proliferated while culturing the cells in the medium for inducing MSC differentiation. In detail, the medium for inducing MSC differentiation was replaced once every 1 day to 4 days without cell washing. At this time, when the cultured cells occupied 80% to 90% of the area of the culture plate, i.e., confluency reached 80% to 90%, subculture was performed. After washing with PBS, subculture was performed by adding TrypLE select (Thermo Fisher) and reacting for 5 minutes under conditions of 37° C. and 5% CO₂. Subsequently, the solution in which TrypLE select and the cells were mixed was centrifuged and resuspended, and then seeded on a dish coated with 0.1% gelatin. Serial passaging was continuously performed to passage 25 using the medium for inducing MSC differentiation, thereby obtaining cells differentiated into mesenchymal stem cells. The cells differentiated into mesenchymal stem cells thus obtained were cultured not on the dish coated with 0.1% gelatin but on a general cell culture dish.

3. Confirmation of Differentiation of Dedifferentiated Stem Cells into Mesenchymal Stem Cells

(3.1) Confirmation of Morphological Changes of Differentiated Cells

Morphological changes of the dedifferentiated stem cells obtained in 1 were examined during differentiation into mesenchymal stem cells in the medium for inducing MSC differentiation.

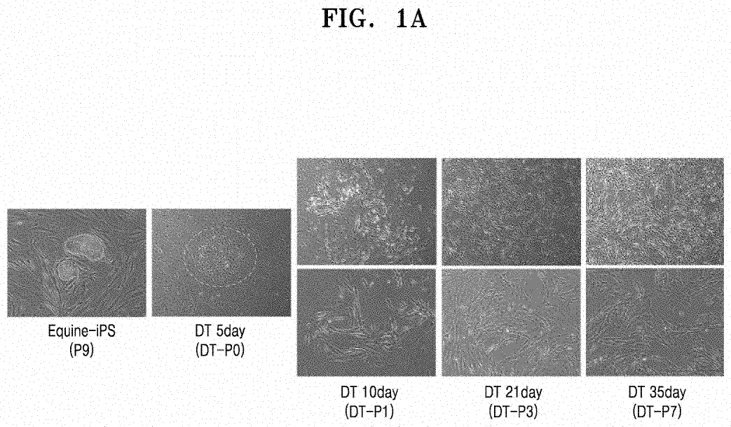

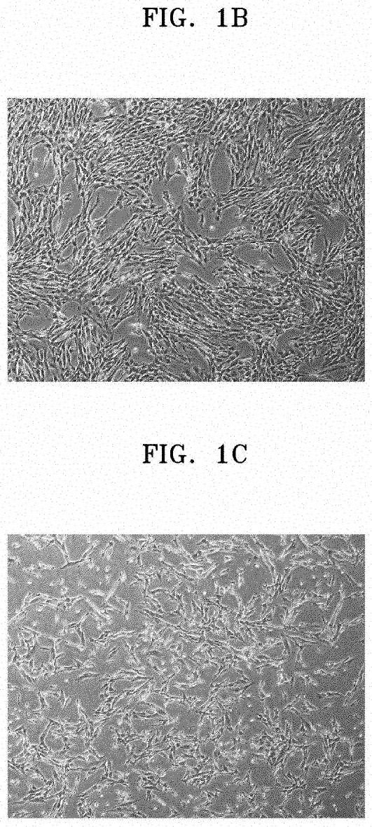

FIG. 1A shows images of optical microscopy of the differentiation process of dedifferentiated stem cells into mesenchymal stem cells. DT represents a period of differentiation into mesenchymal stem cells, and P represents the passage number. FIG. 1B shows images of optical microscopy of differentiated stem cells at 7 passages post-differentiation of dedifferentiated stem cells into mesenchymal stem cells. FIG. 1C shows images of optical microscopy of differentiated stem cells at 14 passages post-differentiation of dedifferentiated stem cells into mesenchymal stem cells. As shown in FIG. 1, on day 5 post-differentiation (early stage of differentiation) of culturing and proliferating equine dedifferentiated stem cells in the medium for inducing MSC differentiation, large round nucleus and small cytoplasm were observed. As the differentiation progressed, shining cells began to appear on day 10 post-differentiation at passage 1. The shining cells had spindle-shaped nuclei and cytoplasm, and showed a high cytoplasmic ratio. Such morphological characteristics of nucleus and cytoplasm were maintained even after subculture. Further, when subculture was performed while culturing in the medium for inducing MSC differentiation, undifferentiated cells and aged cells were removed, and purity of the cells differentiated into mesenchymal stem cells was increased. At passage 7, a large number of shining cells having spindle-shaped nuclei and cytoplasm appeared.

(3.2) Confirmation of CD29 and CD44 Expression in Differentiated Cells

To characterize the mesenchymal stem cells obtained in 2, which were differentiated from dedifferentiated stem cells, mRNA levels of CD29 and CD44 were examined.

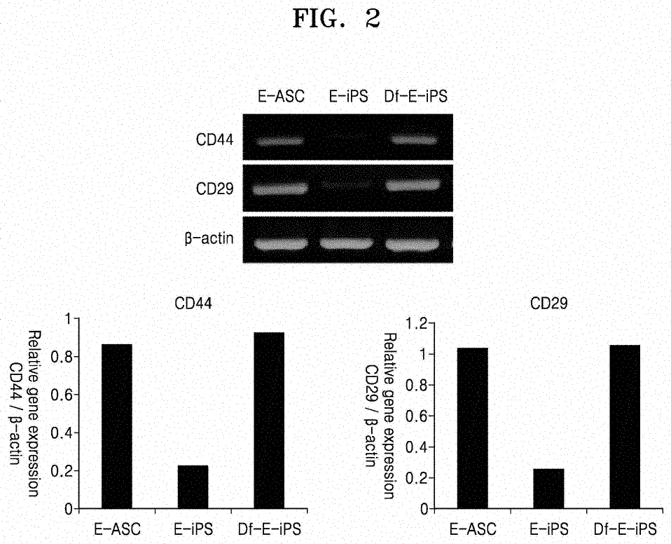

Among the mesenchymal stem cells obtained in 2, mesenchymal stem cells at passage 7 were seeded on a 35 mm dish. When the culture cells occupied about 90% of the area of 35 mm dish, TRIzol and phenol/chloroform were added to the cells to isolate RNA from the cells. Subsequently, reverse transcription of the isolated RNA was performed to synthesize cDNA. Thereafter, RT-PCR was performed using cDNA as a template and a set of primers each specific to CD29 and CD44. Subsequently, PCR products were loaded on a 1.5% agarose gel, followed by electrophoresis. FIG. 2 shows RT-PCR results of examining mRNA levels of CD44 and CD29 in equine dedifferentiated stem cells, equine adipose-derived mesenchymal stem cells, and mesenchymal stem cells differentiated from equine dedifferentiated stem cells. As shown in FIG. 2, no CD29 and CD44 expression was observed in dedifferentiated stem cells, whereas high CD29 and CD44 expression was observed in differentiated mesenchymal stem cells obtained in 2. The expression levels were similar to those of equine adipose-derived stem cells as a positive control.

TABLE 2

| | Forward primer | Reverse primer |
|---|---|---|
| CD44 | ATCCTCACGTCCAACACCCTC | CTCGCCTTTCTGGTGTAGC |
| | (SEQ ID NO: 1) | (SEQ ID NO: 2) |
| CD29 | GATGCCGGGTTTCACTTTGC | TTCCCCTGTTCCATTCACCC |
| | (SEQ ID NO: 3) | (SEQ ID NO: 4) |

(3.3) Confirmation of OCT4 and Nanog Expression in Differentiated Cells

To characterize the mesenchymal stem cells obtained in 2, which were differentiated from dedifferentiated stem cells, mRNA levels of OCT4 and Nanog which are pluripotency markers were examined.

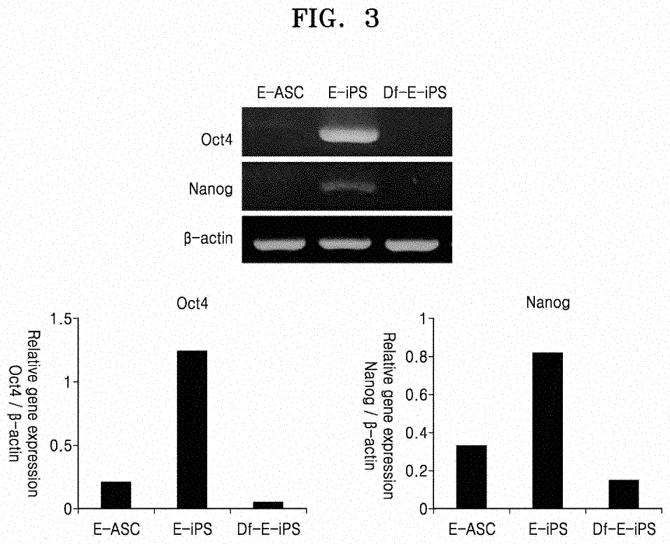

RT-PCR was performed in the same manner as in 3.1., except that a set of primers each specific to OCT4 and Nanog were used. Subsequently, PCR products were loaded on a 1.5% agarose gel, followed by electrophoresis. FIG. 3 shows PCR results of examining mRNA levels of OCT4 and Nanog in equine dedifferentiated stem cells, equine adipose-derived mesenchymal stem cells, and mesenchymal stem cells differentiated from equine dedifferentiated stem cells. As shown in FIG. 3, strong OCT4 and Nanog expression was observed in equine dedifferentiated stem cells, whereas OCT4 and Nanog expression was hardly observed in differentiated mesenchymal stem cells obtained in 2. The expression showed patterns similar to those of equine adipose-derived stem cells. When equine dedifferentiated stem cells were differentiated into mesenchymal stem cells in the medium for inducing differentiation, loss of pluripotency markers was observed.

TABLE 3

| | Forward primer | Reverse primer |
|---|---|---|
| OCT4 | GGGACCTCCTAGTGGGTCA | TGGCAAATTGCTCGAGGTCT |
| | (SEQ ID NO: 5) | (SEQ ID NO: 6) |
| Nanog | TCCTCAATGACAGATTTCAGAGA | GAGCACCAGGTCTGACTGTT |
| | (SEQ ID NO: 7) | (SEQ ID NO: 8) |

(3.4) Confirmation of CD44 and CD29 Expression on Surface of Differentiated Cells

To characterize the mesenchymal stem cells obtained in 2, which were differentiated from dedifferentiated stem cells, CD44 and CD29 expression on the cell surface was examined by flow cytometry.

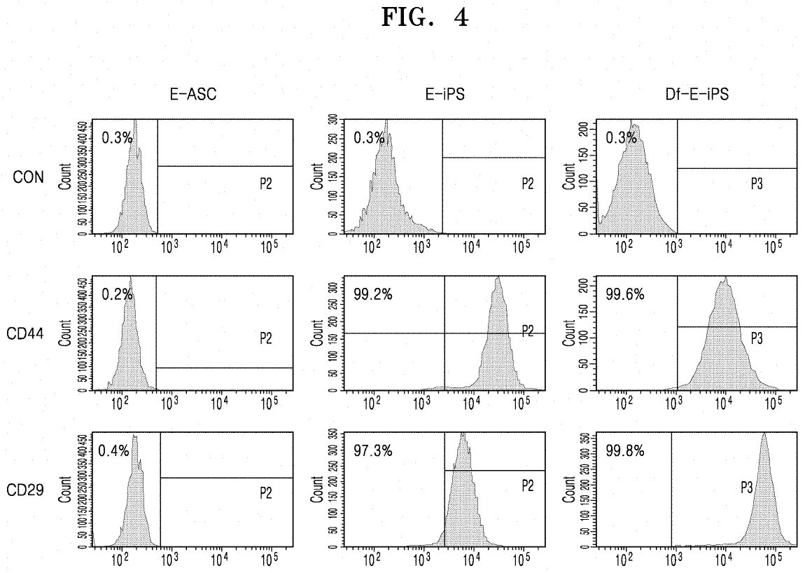

At passage 7 of the mesenchymal stem cells obtained in 2, 1.5×10⁵ cells were suspended in 200 μl of PBS, and 2 μl of anti-human CD44-PE (Phycoerythrin) (eBioscience) as a primary antibody was added and allowed to react at 4° C. for 30 minutes. Subsequently, the cells were centrifuged at 2000 rpm for 5 minutes, and washed with PBS. Then, cell surface markers were examined using BD aria FACS. A group to which the antibody was not added was used as a control. FIG. 4 shows FACS results of examining expression of cell surface marker CD44 in equine dedifferentiated stem cells, equine adipose-derived mesenchymal stem cells, and mesenchymal stem cells differentiated from equine dedifferentiated stem cells. As shown in FIG. 4, equine dedifferentiated stem cells were negative for CD44 whereas 99.6% of the mesenchymal stem cells differentiated from equine dedifferentiated stem cells by the medium for inducing MSC dedifferentiation were positive for CD44. This was similar to the result that 99.2% of equine adipose tissue-derived stromal cells as a positive control were positive for CD44. This was consistent with the previous RT-PCR results in 3.2.

At passage 22 of the mesenchymal stem cells obtained in 2, 1.5×10⁵ cells were suspended in 200 μl of PBS, and 2 μl of anti-mouse CD29-PE (Phycoerythrin) (eBioscience) as a primary antibody was added and allowed to react at 4° C. for 30 minutes. Subsequently, the cells were centrifuged at 2000 rpm for 5 minutes, and washed with PBS. Then, cell surface markers were examined using BD aria FACS. A group to which the antibody was not added was used as a control. FIG. 4 shows FACS results of examining expression of the cell surface marker CD29 in equine dedifferentiated stem cells, equine adipose-derived mesenchymal stem cells, and mesenchymal stem cells differentiated from equine dedifferentiated stem cells. As shown in FIG. 4, the equine dedifferentiated stem cells were negative for CD29 whereas 99.8% of the mesenchymal stem cells differentiated from equine dedifferentiated stem cells by the medium for inducing MSC dedifferentiation were positive for CD29. This was a value of purity similar to or higher than the result that 97.3% of equine adipose tissue-derived stromal cells as a positive control were positive for CD29. This was also consistent with the previous RT-PCR results in 3.2.

1. A medium for inducing differentiation of dedifferentiated stem cells into mesenchymal stem cells, the medium comprising glucose, insulin, selenium, transferrin, and vascular endothelial growth factor (VEGF).

2. The medium for inducing differentiation of claim 1, wherein the dedifferentiated stem cells are derived from adipose tissue, bone marrow, umbilical cord blood, or a placenta.

3. The medium for inducing differentiation of claim 1, wherein the dedifferentiated stem cells are derived from a horse, a dog, a cat, a fetus a calf, a human, or a mouse.

4. The medium for inducing differentiation of claim 1, wherein the glucose is included in an amount of 100 mg/L to 10000 mg/L, the insulin is included in an amount of 0.3 mg/L to 30 mg/L, the transferrin is included in an amount of 0.27 mg/L to 27 mg/L, the selenium is included in an amount of 0.0000003 mg/L to 0.00003 mg/L, and the VEGF is included in an amount of 0.001 mg/L to 0.1 mg/L.

5. The medium for inducing differentiation of claim 1, comprising biotin and niacin.

6. The medium for inducing differentiation of claim 5, wherein the biotin is included in an amount of 0.01 mg/L to 1.0 mg/L and the niacin is included in an amount of 0.1 mg/L to 10 mg/L.

7. A method of preparing mesenchymal stem cells from dedifferentiated stem cells, the method comprising:
introducing a dedifferentiation inducer protein or a polynucleotide that encodes them into isolated somatic cells or isolated adult stem cells to induce dedifferentiation of stem cells from the isolated somatic cells or the isolated adult stem cells; and
culturing the induced dedifferentiated stem cells in the medium for inducing differentiation of claim 1 to induce differentiation of mesenchymal stem cells from dedifferentiated stem cells.

8. The method of claim 7, wherein the dedifferentiated stem cells are derived from adipose tissue, bone marrow, umbilical cord blood, or a placenta.

9. The method of claim 7, wherein the dedifferentiated stem cells are derived from a horse, a dog, a cat, a fetus, a calf, a human, or a mouse.

10. The method of claim 7, wherein the inducing of differentiation into mesenchymal stem cells is to perform subculture for 1 passage to 25 passages.

11. The method of claim 7, wherein the inducing of differentiation into mesenchymal stem cells is cultured for 2 days to 80 days.

12. A mesenchymal stem cell prepared by the method of claim 7.

13. The mesenchymal stem cell of claim 12, wherein the mesenchymal stem cell has surface antigenic characteristics of CD29⁺ and CD44⁺.
